(19) United States
(12) Patent Application Publication
(10) Pub. No.: US 2025/0151986 A1
(43) Pub. Date: May 15, 2025

(54) MEDICAL SYSTEMS, DEVICES, AND RELATED METHODS FOR COUPLING MEDICAL DEVICES

(71) Applicant: Boston Scientific Scimed, Inc., Maple Grove, MN (US)

(72) Inventors: Austin Grant JOHNSON, Worcester, MA (US); Colby HARRIS, Norfolk, MA (US)

(73) Assignee: Boston Scientific Scimed, Inc., Maple Grove, MN (US)

(21) Appl. No.: 18/941,845

(22) Filed: Nov. 8, 2024

Related U.S. Application Data
(60) Provisional application No. 63/599,029, filed on Nov. 15, 2023.

(51) Int. Cl. A61B 1/00; A61B 1/018

(52) U.S. Cl. CPC A61B 1/0014; A61B 1/018

(57) ABSTRACT

A coupling device for a medical system includes a first portion and a second portion. The first portion includes a main body and a connector. The main body has a device receiving portion that includes a first slot and an opening. A portion of the opening includes an internal threading. The connector extends from the main body and is configured to couple a first medical device to the first portion. The second portion includes a second slot configured to receive a portion of a second medical device. The second portion includes a threaded portion. The first portion and the second portion are removably couplable by coupling the threaded portion of the second portion to the internal threading of the first portion to removably couple the second medical device to the first medical device.

### CROSS-REFERENCE TO RELATED APPLICATIONS

This application claims the benefit of priority under 35 U.S.C. § 119 from U.S. Provisional Application No. 63/599,029, filed Nov. 15, 2023, which is incorporated by reference herein in its entirety.

### TECHNICAL FIELD

This disclosure generally relates to medical systems, devices, and related methods that may be used to treat a subject. Aspects of the disclosure relate to medical systems, devices, and methods for coupling (e.g., releasably coupling) medical devices, for example, for endoscopic medical procedures, such as cutting, capturing, or otherwise manipulating tissue, objects, or material with one or more medical devices, among other aspects.

### BACKGROUND

Various medical techniques and instruments have been developed for diagnosis and/or treatment within a patient's body, such as within a patient's gastrointestinal (GI) tract or within a patient's urinary system. Endoscopic sub-mucosal dissection (ESD), endoscopic sub-mucosal resection (ESR), mucosal resection (EMR), polypectomy, mucosectomy, etc., are examples of minimally invasive treatment methods for both malignant and non-malignant lesions. Such procedures often require multiple endoscopic instruments for the resection of tissue. In some instances, a secondary medical device may be inserted through a portion of a primary medical device. A second user may be necessary to hold and/or manipulate the secondary medical device, with the first user holding and/or manipulating the primary medical device. In some instances, the secondary medical device may be coupled to the primary medical device, which may allow the first user to hold and/or manipulate both the primary medical device and the secondary medical device. However, different sized and/or shaped secondary medical devices may require different sizes and/or shapes of coupling devices in order to securely couple the secondary medical device to the primary medical devices. Such limitations may increase user fatigue, impede the manipulation and/or articulation of one or more of the primary medical device and/or the second medical device, or otherwise affect the procedure.

These concerns may increase the duration, costs, and risks of the medical procedure. The systems, devices, and methods of this disclosure may rectify some of the deficiencies described above or address other aspects of the art.

### SUMMARY

Examples of the disclosure relate to, among other things, systems, devices, and methods for performing one or more medical procedures, for example, endoscopic medical procedures, such as cutting, capturing, or otherwise manipulating tissue, objects, or material with one or more medical devices. Each of the examples disclosed herein may include one or more of the features described in connection with any of the other disclosed examples.

In some aspects, a coupling device for a medical system may include a first portion and a second portion. The first portion may include a main body and a connector. The main body may have a device receiving portion that may include a first slot and an opening. A portion of the opening may include an internal threading. The connector may extend from the main body and may be configured to couple a first medical device to the first portion. The second portion may include a second slot configured to receive a portion of a second medical device. The second portion may include a threaded portion. The first portion and the second portion may be removably couplable by coupling the threaded portion of the second portion to the internal threading of the first portion to removably couple the second medical device to the first medical device.

The coupling device may include one or more of the following aspects. The coupling device may further include a gasket positioned within the opening of the first portion. The gasket may include a gasket slot extending longitudinally through a portion of the gasket. The gasket may be configured to transition between at least a first configuration and a second configuration. In the first configuration, the gasket slot may include a first width, and, in the second configuration, the gasket slot may include a second width that is less than the first width. The gasket may include a gasket opening configured to receive a portion of the second medical device. The second portion may include a grip portion adjacent to the threaded portion. The second portion may include an end portion adjacent to the grip portion on an opposite side from the threaded portion, and the end portion may be wider than the grip portion. The second slot may extend longitudinally through the end portion, the grip portion, and the threaded portion of the second portion.

The connector may extend from a first end of the main body of the first portion, and the device receiving portion may extend from a second end of the main body opposite to the first end. The main body may include a connector receiving portion that may include an opening and two extensions, with the opening positioned between the two extensions. The connector may include a narrowed portion and a widened portion, and the narrowed portion may be configured to be received within the opening of the connector receiving portion to couple the first portion of the coupling device to the first medical device. The connector may be formed of an at least partially flexible or elastic material. The widened portion of the connector may include a grip surface. The device receiving portion may be rotatable relative to the main body of the first portion. The first portion may be configured to be coupled to an insertion portion of the first medical device, and the second portion may be configured to be coupled to an insertion portion of the second medical device.

In another aspect, a medical system may include a first medical device, a second medical device, and a coupling device. The first medical device may include a first handle and a first insertion portion, and the first handle may include a port. The second medical device may include a second handle and a second insertion portion, and at least a portion of the second insertion portion may be configured to be inserted into the port. The coupling device may include a first portion, a gasket, and a second portion. The first portion may include a main body and a connector. The main body may include a device receiving portion. The device receiving portion may include a first slot and an opening, and a portion of the opening may include an internal threading. The connector may extend from the main body and may be configured to couple a first medical device to the first portion. The gasket may be positioned within the opening of the first portion, and the gasket may include a gasket slot aligned with the slot in the device receiving portion. The second portion may include a second slot configured to receive a portion of a second medical device, and the second portion may include a threaded portion. The first portion and the second portion may be removably couplable by coupling the threaded portion of the second portion to the internal threading of the first portion to (1) removably couple the second medical device to the first medical device, and (2) reduce a size of the gasket.

The medical system may include one or more of the following aspects. The second portion may include (1) a grip portion adjacent to the threaded portion, and (2) an end portion adjacent to the grip portion that is on an opposite side from the threaded portion and is wider than the grip portion. The second slot may extend longitudinally through the end portion, the grip portion, and the threaded portion. The device receiving portion of the first portion may be rotatable relative to the main body of the first portion.

In yet another aspect, a coupling device may include a first portion, a gasket, and a second portion. The first portion may include a main body and a connector. The main body may include a device receiving portion that includes a first slot and an opening. The connector may extend from the main body and may be configured to couple a first medical device to the first portion. The gasket may be positioned within the opening of the first portion, and the gasket may include a gasket slot aligned with the slot in the device receiving portion. The second portion may include a second slot configured to receive a portion of a second medical device. The first portion and the second portion may be removably couplable to removably couple the second medical device to the first medical device. The first slot and the opening in the device receiving portion of main body of the first portion may both be configured to reduce in size to secure the second portion to the first portion.

The coupling device may include one or more of the following aspects. The device receiving portion may be rotatable relative to the main body of the first portion.

It may be understood that both the foregoing general description and the following detailed description are exemplary and explanatory only and are not restrictive of this disclosure, as claimed.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 illustrates a medical system, including a primary medical device, a secondary medical device, and a coupling device, according to aspects of this disclosure.

FIG. 2 illustrates the coupling device in an uncoupled configuration, according to aspects of this disclosure.

FIG. 3A illustrates a portion of the coupling device in a first, uncompressed configuration, and FIG. 3B illustrates the portion of the coupling device in a second, compressed configuration, according to aspects of this disclosure.

FIGS. 4A-4D illustrate the medical system of FIG. 1, including the primary medical device, the secondary medical device, and the coupling device, in various configurations, according to aspects of this disclosure.

FIG. 5 illustrates another coupling device, including an enlarged view of a portion of the another coupling device, according to aspects of this disclosure.

### DETAILED DESCRIPTION

The terms “proximal” and “distal” are used herein to refer to the relative positions of the components of an exemplary medical system and exemplary medical devices. When used herein, “proximal” refers to a position relatively closer to the exterior of the body of a subject or closer to a medical professional using the medical system or medical device. In contrast, “distal” refers to a position relatively further away from the medical professional using the medical system or medical device, or closer to the interior of the body of the subject. Proximal and distal directions are labeled with arrows marked “P” and “D”, respectively, throughout various figures. As used herein, the terms “comprises,” “comprising,” “has,” “having,” “includes,” “including,” or other variations thereof, are intended to cover a non-exclusive inclusion, such that a system, device, or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value.

Embodiments of this disclosure include systems, devices, and methods for manipulating, cutting, grabbing, ligating, biopsying, stenting, controlling bleeding, and/or otherwise treating tissue or a treatment site. In some examples, the systems, devices, and/or methods discussed herein may be utilized during endoscopic mucosal resection (EMR) and/or endoscopic submucosal dissection (ESD) procedures. In examples, EMR and ESD include endoluminal placement of one or more devices for grasping and cutting tissue proximate to a target area within the body of a patient. Placement of the one or more medical devices may be via a catheter, scope (endoscope, bronchoscope, colonoscope, gastroscope, duodenoscope, etc.), tube, or sheath, inserted into the GI tract via a natural orifice or incision. The orifice can be, for example, the nose, mouth, or anus, and the placement can be in any portion of the GI tract, including the esophagus, stomach, duodenum, large intestine, or small intestine. Placement also can be in other organs reachable via the GI tract. In other aspects, the one or more medical devices may be delivered via a catheter, scope (e.g., ureteroscope), tube, or sheath, inserted into the urinary system via a natural orifice (e.g., urethra) or incision. The patient's tissue may be grasped using suction from one or more medical devices and/or a grasper, and then the tissue may be cut by a cutting device for subsequent removal from the patient's body.

In examples, the devices, systems, and/or methods discussed herein may be utilized during natural orifice transluminal endoscopic surgery (NOTES) procedures. In a NOTES procedure, a stent or other device may be delivered in order to create an opening between two body lumens (e.g., a stomach and a small bowel). In examples, the devices, systems, and/or methods discussed herein may be utilized during an endoscopic retrograde cholangiopancreatography (ERCP) procedure, a peroral endoscopic myotomy (POEM) procedure, a hemostasis procedure, a pulmonary nodule biopsy procedure, or other procedures.

Although EMR, ESD, and NOTES are discussed herein, the disclosure is not so limited. Embodiments of the disclosure include systems and devices that may be used (and methods that may be performed) in any suitable procedure in any body lumen or organ.

Reference will now be made in detail to examples of this disclosure described above and illustrated in the accompanying drawings. Wherever possible, the same reference numbers will be used throughout the drawings to refer to the same or like parts.

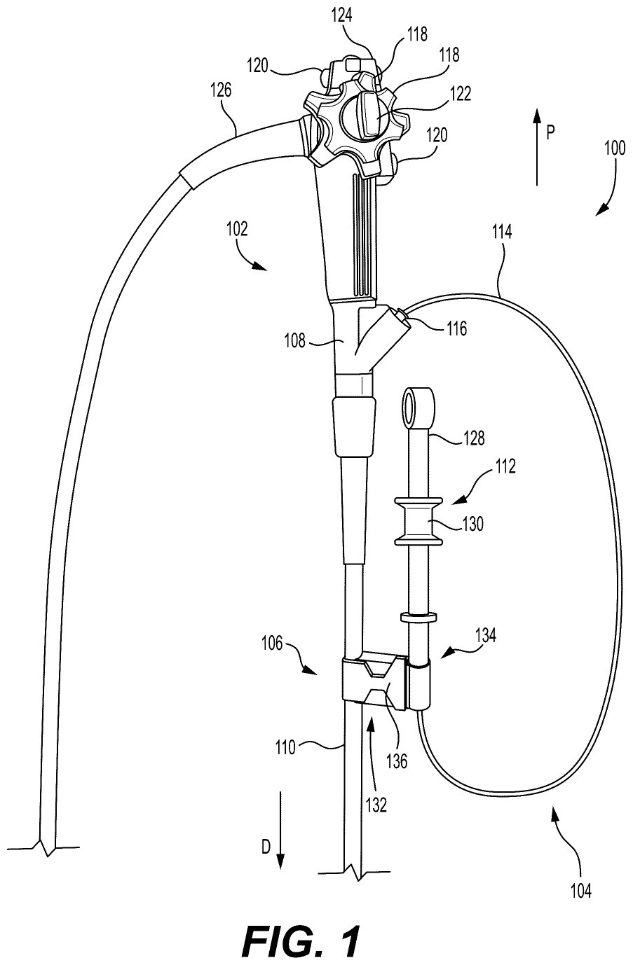

FIG. 1 illustrates an exemplary medical system 100, including a primary or first medical device 102 and a secondary or second medical device 104. First medical device 102 and second medical device 104 may be coupled (e.g., releasably coupled) via a coupling device 106. First medical device 102 includes a handle 108 and an insertion portion 110, and second medical device 104 includes a handle 112 and an insertion portion 114. In some aspects, handle 112 of second medical device 104 may be coupled (e.g., removably coupled0 to first medical device 104 (e.g., to handle 108 and/or insertion portion 110) of via coupling device 106. Moreover, insertion portion 110 of first medical device 102 may be inserted into a subject, and insertion portion 114 of second medical device 104 may be delivered through a portion of insertion portion 110, for example, via a port 116 or other opening in handle 108 of first medical device 102.

As discussed in detail below, coupling device 106 may allow for second medical device 104 (e.g., handle 112 and/or insertion portion 114) to be coupled to handle 108 and/or insertion portion 110 of first medical device 102. Coupling device 106 may allow for second medical device 104 to be coupled to and uncoupled from (e.g., fully detached and separated from) handle 108 and/or insertion portion 110 of first medical device 102. Moreover, coupling device 106 may allow for different second medical devices 104, for example, medical devices that include insertion portions of different sizes and/or shapes, to be coupled to and uncoupled from handle 108 and/or insertion portion 110 of first medical device 102.

First medical device 102 includes handle 108 and insertion portion 110, which includes a distal end (not shown). One or more lumens (e.g., a working channel and fluidics lumens) may extend from handle 108 to the distal end, for example, through insertion portion 110. As mentioned, handle 108 includes port 116 or another opening, which may connect, through handle 108, to the one or more lumens in insertion portion 110. Moreover, handle 108 may include one or more knobs 118 and one or more actuators 120. For example, knobs 118 may be movable (rotatable or pivotable) to control the movement (e.g., deflection) of a distal portion of insertion portion 110. Handle 108 may also include one or more locking devices 122, for example, to secure the position of one or more knobs 118, and thus at least partially secure the position of the distal portion of insertion portion 110. Additionally, actuators 120 may include one or more buttons or other types of actuators. Actuators 120 may be controlled to actuate the delivery of fluid, to actuate the application of suction, to actuate one or more illumination or imaging devices, etc. Furthermore, handle 108 may include one or more elevator actuator 124, for example, to control the position of an elevator positioned within the lumen of insertion portion 110.

First medical device 102 may include or be coupled to an umbilicus 126. For example, umbilicus 126 may be coupled to a proximal portion of handle 108. Umbilicus 126 may connect first medical device 102 to one or more of a control unit, a fluid source, a suction source, and/or other exterior devices, such as a monitor for viewing images from a camera (e.g., at the distal end of insertion portion 110).

Second medical device 104 includes handle 112 and insertion portion 114, and insertion portion 114 may be inserted through port 116. Additionally, handle 112 of second medical device 104 may include a stationary portion 128 and a movable portion 130. In these aspects, movement of movable portion 130 relative to stationary portion 128 may control one or more aspects of second medical device 104. For example, second medical device 104 may include one or more end effectors, and movement of movable portion 130 may control the position, extension, configuration, etc. of the one or more end effectors. Additionally or alternatively, although not shown, handle 112 may include one or more controls similar to knob(s) 118, actuator(s) 120, locking device(s) 122, or elevator actuator(s) 124, and insertion portion 114 may define one or more lumens for receiving other medical devices. The structure of handle 112 is not limited, and handle 112 may include any features of medical device handles known in the art.

In some aspects, first medical device 102 may be a gastroscope. Alternatively, first medical device 102 may be one of other similar insertion devices with a handle, such as, for example, endoscopes, ureteroscopes, duodenoscopes, endoscopic ultrasonography (“EUS”) scopes, colonoscopes, bronchoscopes, laparoscopes, arthroscopes, cystoscopes, aspiration scopes, etc. Moreover, second medical device 104 may be a stent delivery device, for example, with a stent releasably positioned at a distal end of insertion portion 114, such that manipulation of handle 112 (e.g., of movable portion 130) controls the deployment, delivery, or positioning of the stent. Alternatively, second medical device 104 may be one of other end effector devices, such as, for example, graspers, clamps, clips, snares, electrosurgical knives or other electrocautery devices, retrieval devices, biopsy needles, hemostatic patch delivery devices, hemostatic spray delivery devices, etc. Second medical device 104 alternatively may be a scope, such as a cholangioscope.

Coupling device 106 includes a first portion 132 and a second portion 134. Coupling device 106 also includes a connector or strap 136, for example, coupled to first portion 132. Strap 136 may be configured to wrap around a portion of handle 108 and/or insertion portion 110 of first medical device 102 to releasably secure coupling device 106 to handle 108. Additionally, second portion 134 of coupling device 106 may be movable relative to first portion 132 of coupling device 106, for example, to couple coupling device 106 to a portion of second medical device 104, for example, to insertion portion 114.

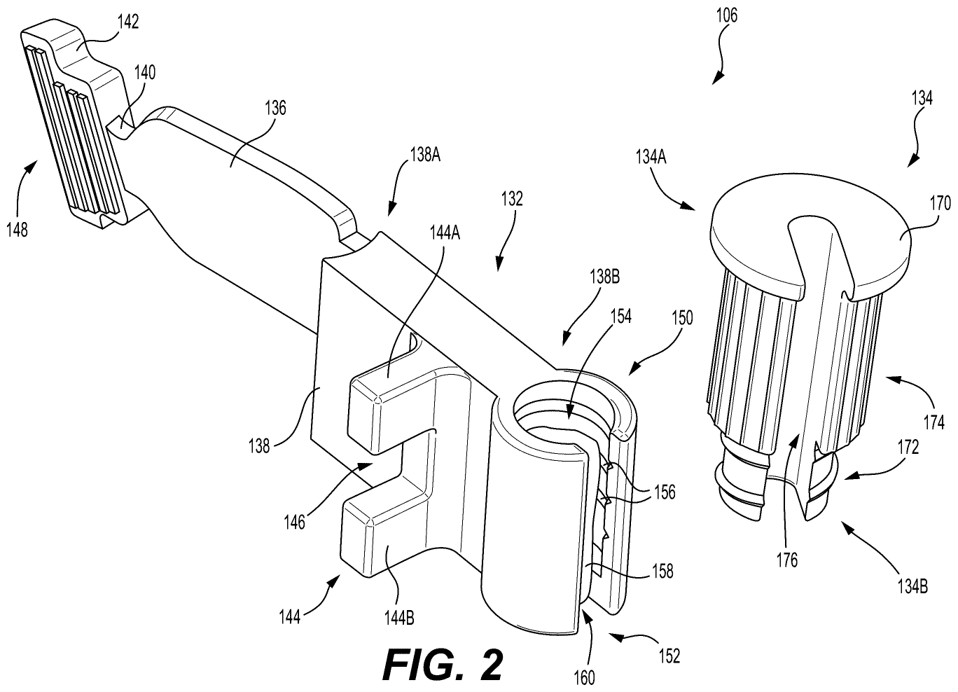

FIG. 2 illustrates coupling device 106 separate from first medical device 102 and second medical device 104. As shown, first portion 132 may include strap 136 and a main body 138. One end of strap 136 may be fixedly coupled a portion of main body 138, for example, at one end or a first end 138A of main body 138. Another end of strap 136 may include a narrowed portion 140 and a widened portion 142, for example, with the widened portion 142 at the another end opposite to the coupling of strap 136 to main body 138. Additionally, a portion of main body 138 may include a connector or strap receiving portion 144, for example, including an opening 146 formed between two extensions 144A, 144B. In these aspects, the narrowed portion 140 of strap 136 may be positioned within opening 146 of strap receiving portion 144 to couple (e.g., removably couple) strap 136 to main body 138.

As strap 136 may be wrapped around a portion (e.g., handle 108) of first medical device 102, coupling narrowed portion 140 of strap 136 to strap receiving portion 144 of main body 138 may couple at least a portion of coupling device 106 (e.g., first portion 132) to first medical device 102. Additionally, widened portion 142 may help to securely couple strap 136 to main body 138. In some aspects, as shown in FIG. 2, one or more portions of widened portion 142 may include raised and/or depressed sections, for example, forming a grip surface 148. Strap 136 may thus be utilized to removably couple first portion 132 to first medical device 102, for example, to primary medical device(s) of different sizes and/or shapes. Alternatively, although not shown, first portion 132 may be fixedly coupled to first medical device 102 (e.g., integrated into and integrally formed with handle 108).

First portion 132 includes a device receiving portion 150, for example, to receive a portion of second medical device 104 and/or second portion 134. Device receiving portion 150 may be positioned on a second end 138B of main body 138, for example, on an opposing side of main body 138 from first end 138A. Device receiving portion 150 may be partially cylindrical. Specifically, device receiving portion 150 includes a slot 152 and an opening 154. As shown, slot 152 extends longitudinally through a length of an outer or exterior portion of device receiving portion 150. Opening 154 extends through device receiving portion 150, for example, forming a hollow, at least partially cylindrical portion within device receiving portion 150. For example, opening 154 and slot 152 may define a lumen that has an opening on a radial side of the lumen, such that a side of the lumen is open via slot 152. In some aspects, a portion (e.g., a top portion in FIG. 2) of opening 154 includes an internal grooving or threading 156.

Additionally, first portion 132 includes a compressible element or a gasket 158. Gasket 158 may be sized and configured to be positioned within opening 154 of device receiving portion 150, for example, with a cross-section that is at least partially circular, rectangular, or otherwise sized to be positioned within opening 154 of device receiving portion 150. Moreover, gasket 158 includes a slot, or a gasket slot 160, for example, at least partially overlapping with slot 152. Gasket 158 is at least partially flexible or compressible, for example, like a Tuohy Borst gasket, and gasket 158 may compress or otherwise secure a portion of second medical device 104 when coupling device 106 secures second medical device 104 to first medical device 102. For example, gasket 158 may help to shrink or otherwise reduce an inner diameter of opening 154 when first portion 132 reduces in size.

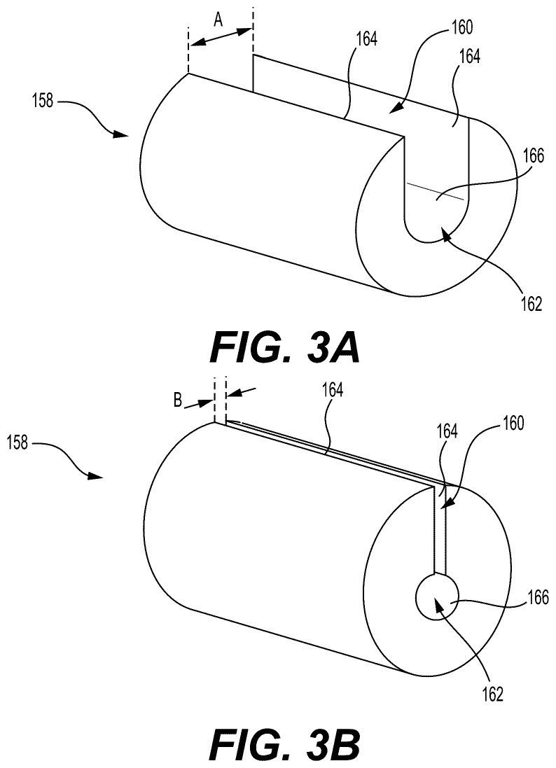

In some aspects, gasket 158 may be formed of a rubber, rubber-based, or otherwise compressible or malleable material (e.g., a compliant or semi-compliant material, a plastic, silicone, rubber, etc.). Alternatively or additionally, gasket 158 may be a U-ring, for example, like an O-ring but with gasket slot 160 interrupting an outer circumference or shape of gasket 158. In these aspects, gasket 158 is partially circular, including gasket slot 160, and is at least partially compressible. As discussed below and as shown in FIG. 3B, when gasket 158 is compressed, gasket slot 160 may reduce in size and/or form a gasket opening 162, for example, extending longitudinally through a central portion of gasket 158. In these aspects, gasket 158 may help to couple secondary medical devices of different sizes and/or shapes to coupling device 106, and thus to a primary medical device.

Moreover, second portion 134 may form a twist knob and/or otherwise be couplable to first portion 132 of coupling device 106, for example, to help reduce the overall size of first portion 132. Second portion 134 includes a first end 134A and a second end 134B opposite to the first end 134A. First end 134A may include an end portion 170. Second end 134B may include a threaded portion 172, for example, corresponding to the internal grooving or threading 156 within opening 154 of device receiving portion 150. Additionally, second portion 134 may include a grip portion 174 (e.g., including one or more indentations or extensions) extending between the end portion 170 and threaded portion 172. Threaded portion 172 may include a smaller cross-sectional size than grip portion 174, and grip portion 174 may include a smaller cross-sectional size than end portion 170, such that end portion 170 is wider than grip portion 174. Additionally, the various portions or sections of second portion 134 may be generally cylindrical, albeit with a slot 176 extending longitudinally through second portion 134 (e.g., from top to bottom as shown in FIG. 2). Slot 176 may receive a portion of second medical device 104 (e.g., handle 112 and/or insertion portion 114). In some configurations, slot 176 of second portion 134 may at least partiallyalign with slot 152 in device receiving portion 150. Additionally, gasket 158 may also be positioned within device receiving portion 150, such that gasket slot 160 at least partially aligns with slot 152 in device receiving portion 150 and, correspondingly, at least partially aligns with slot 176 of second portion 134.

Various aspects of first portion 132 and second portion 134 of coupling device 106 may be formed of various materials. For example, main body 138 of first portion 132 may be formed of a plastic or metallic material. Strap 136 of first portion 132 may be at least partially elastic or flexible, for example, formed of a plastic, silicone, rubber, etc. As such, strap 136 may be at least partially flexible or stretchable to stretch around a portion of first medical device 102, be coupled to main body 138, and then tighten around the portion of first medical device 102 to secure first portion 132 of coupling device 106 to first medical device 102. Second portion 134 may also be formed of a plastic or metallic material, for example, such that second portion 134 is at least partially rigid. Gasket 158 may be formed any compliant or semi-compliant material, for example, a plastic, silicone, rubber, etc., that allows gasket 158 to be at least partially compressed to reduce the inner diameter. In these aspects, gasket 158 may reduce in size and help secure a portion of second medical device 104 within coupling device 106, and thus couple second medical device 104 to first medical device 102.

Additionally, although not shown, first portion 132 of coupling device 106 may include one or more other features that allow the coupling device 106 to be attached to first medical device 102. For example, instead of strap 136 being at least partially flexible or elastic, coupling device 106 may include one or more of a zip tie, a slap bracelet-style closure, a twist tie, or a specific geometry or device that mates with or is otherwise couplable with one or more portions of first medical device 102. In these aspects, coupling device 106 may be coupled to handle 108 and/or insertion portion 110 of first medical device 102.

FIG. 3A illustrates gasket 158 in a first or uncompressed state, and FIG. 3B illustrates gasket 158 in a second or compressed state. As shown in FIG. 3A, in the first or uncompressed state, gasket 158 includes gasket slot 160, with gasket slot 160 including a first width A. As shown, gasket slot 160 may include a substantially U-shaped cross-section, for example, with edges 164 and a curved bottom or inner portion 166. The size of gasket slot 160 in the first or uncompressed state may substantially overlap with slot 152 in device receiving portion 150 of first portion 132. For example, gasket opening 162 form a substantially U-shaped cross-section in the first state, forming inner portion 166, as shown in FIG. 3A. Furthermore, edges 164 may help to form gasket slot 160.

As shown in FIG. 3B, in the second or compressed state, gasket 158 may include a smaller gasket slot 160, for example, including a second width B that is smaller than first width A. In some aspects, opposing edges 164 of gasket slot 160 may be in abutting contact when gasket 158 is in the second or compressed state. Nevertheless, in the second or compressed state, gasket 158 includes gasket opening 162, for example, an at least partially circular opening in a central portion of gasket 158.

As discussed in detail below, a portion of second medical device 104 may be positioned within gasket slot 160 when gasket 158 is in the first or uncompressed state, for example, in bottom or inner portion 166 of gasket slot 160/opening 162. Then, gasket 158 may be transitioned from the first or uncompressed state to the second or compressed state, such that the portion of second medical device 104 is at least partially secured within gasket opening 162. Additionally, the size of gasket slot 160 in the second or compressed state (e.g., second width B) may be smaller than a width or size of the portion of second medical device 104. As such, gasket 158 may help to retain second medical device 104 within coupling device 106 when gasket 158 is in the second or compressed state. Additionally, as mentioned, gasket 158 is at least partially flexible, so gasket 158 may accommodate different sizes and/or shapes of second medical device 104, or other secondary medical device(s).

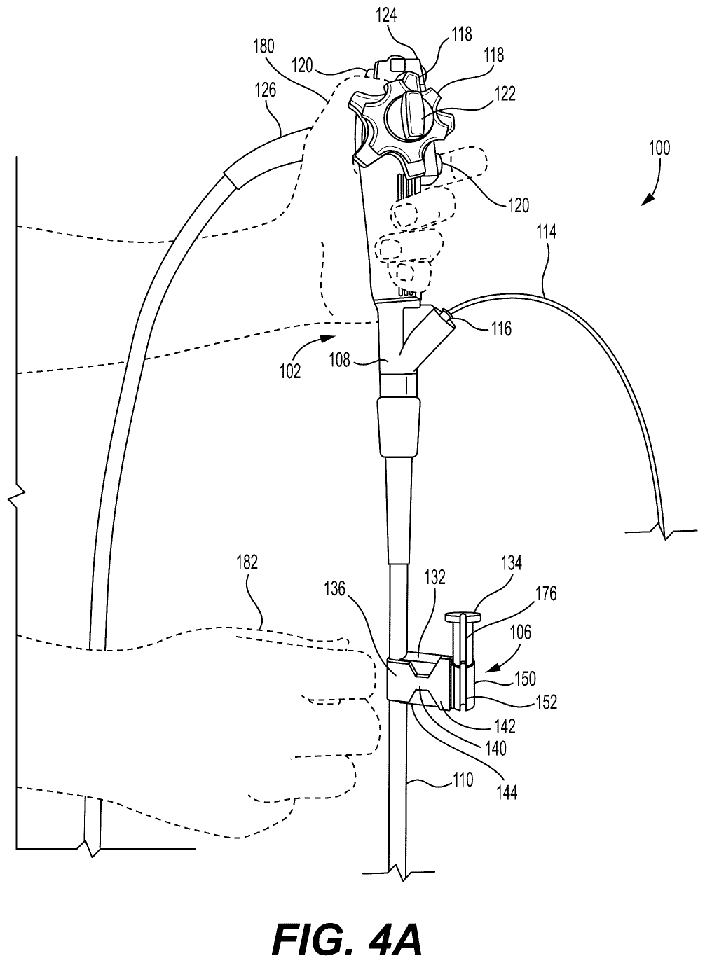

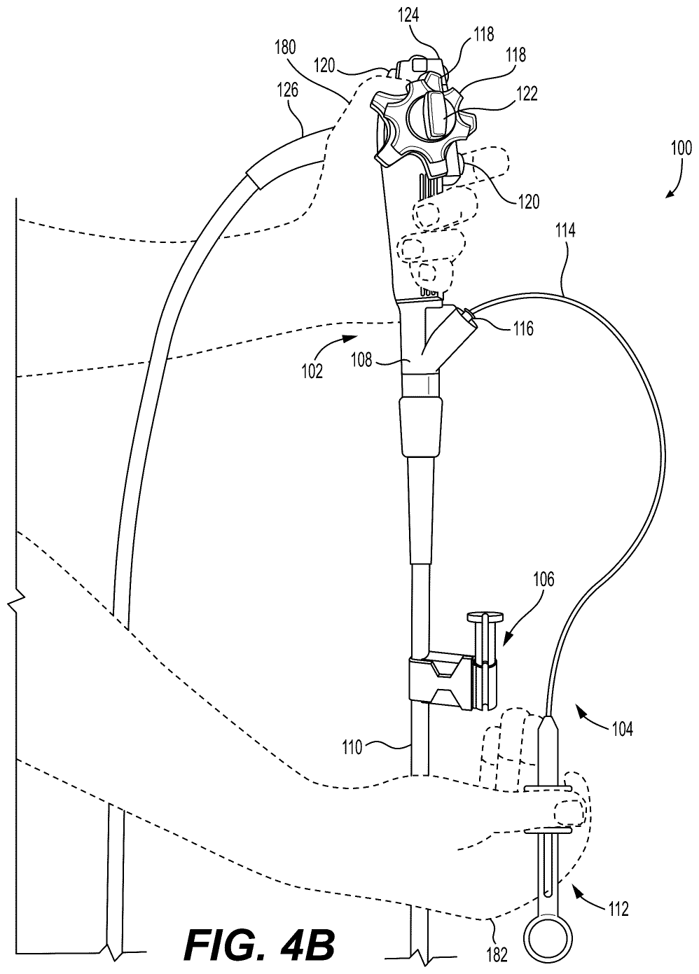

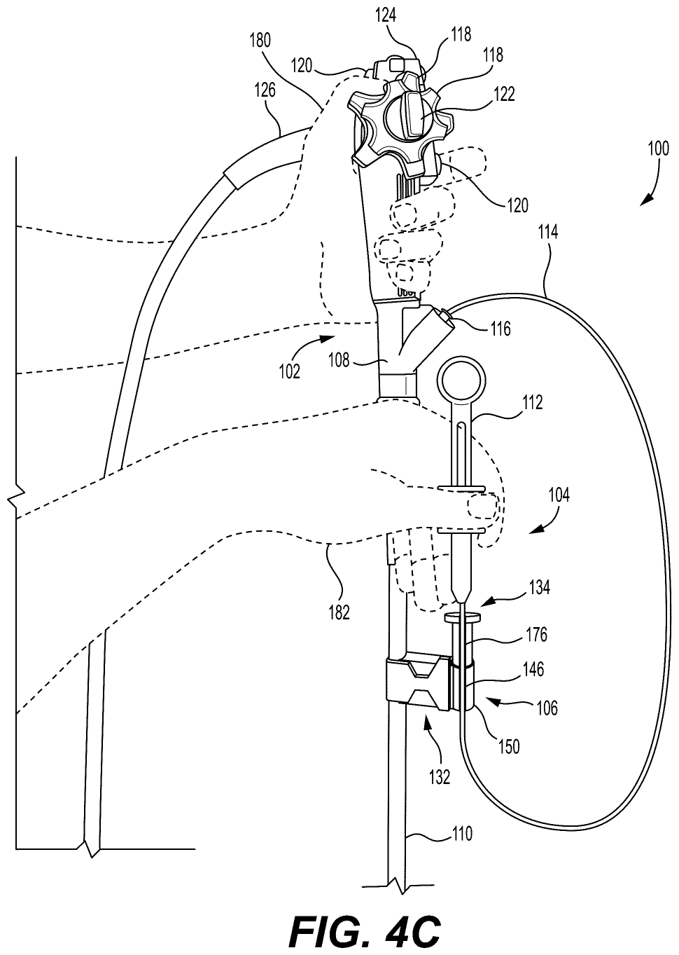

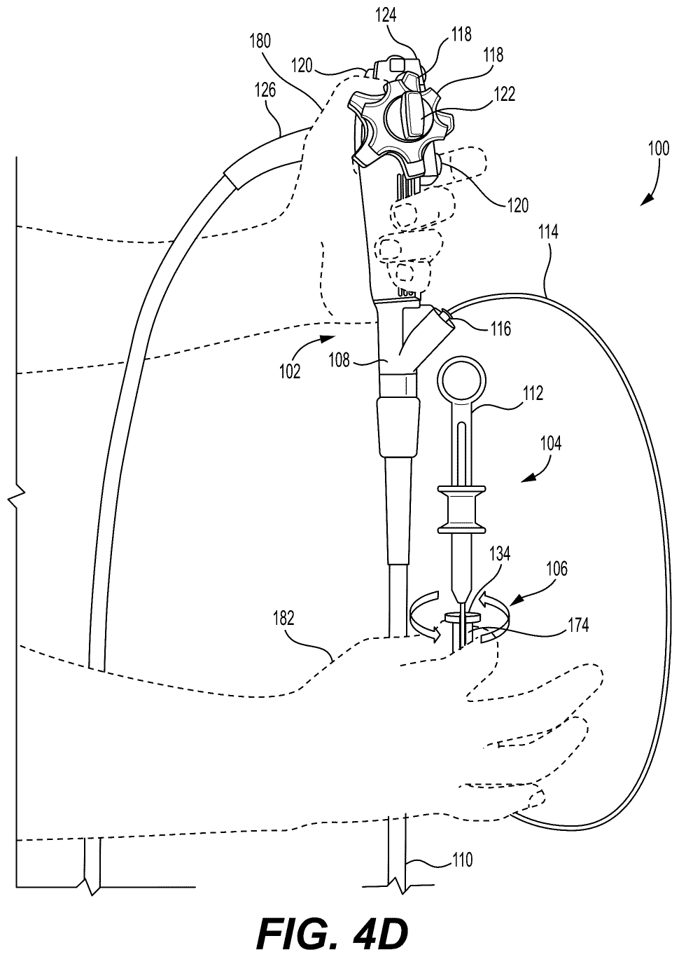

FIGS. 4A-4D illustrate various steps of coupling second medical device 104 to first medical device 102 via coupling device 106, for example, with hands 180, 182 of a user. As shown in FIG. 4A, first portion 132 of coupling device 106 may be coupled to a portion of first medical device 102, for example, to a portion of insertion portion 110. For example, as discussed above, strap 136 may be wrapped around a portion of insertion portion 110. Strap 136 may be secured to main body 138, for example, with narrowed portion 140 and widened portion 142 interacting with strap receiving portion 144. The user may manipulate strap 136 with hand 182, while the user maintains the position of first medical device 102 with hand 180. Additionally, in some aspects, second portion 134 may be coupled to first portion 132 in a first configuration, for example, with threaded portion 172 (FIG. 2) being partially received within threading 156 (FIG. 2) of device receiving portion 150. Moreover, in this configuration, slot 152, gasket slot 160 (FIGS. 3A and 3B), and slot 176 may be at least partially aligned. Insertion portion 114 of second medical device 104 may be delivered through a portion of first medical device 102, for example, through port 116 in handle 108.

Next, as shown in FIG. 4B, a portion of handle 112 of second medical device 104 may be positioned adjacent to coupling device 106. For example, as shown, with a portion of insertion portion 114 positioned within port 116, the user may control, manipulate, or otherwise hold handle 112 with hand 182. The user may maintain the position of first medical device 102 with hand 180. The user may position second medical device 104, for example, a proximal portion of insertion portion 114 based on the movement of handle 112 via hand 182.

Furthermore, as shown in FIG. 4C, a portion of insertion portion 114 (e.g., a proximal portion of insertion portion 114) may be positioned within a portion of coupling device 106. For example, insertion portion 114 may be positioned within slot 152, gasket slot 160 (FIGS. 3A and 3B), and slot 176, such that a portion of second medical device 104 is coupled to device receiving portion 150. Handle 112 of second medical device 104 may be positioned adjacent to handle 108 of first medical device 102. For example, as shown in FIG. 4C, insertion portion 110 of first medical device 102 may extend downward from handle 108, and insertion portion 114 of second medical device 104 may extend downward from handle 112. Alternatively, although not shown, second medical device 104 may be coupled to first medical device 102 in an opposite configuration, for example, with handle 112 adjacent to insertion portion 110 and such that handle 112 extends downward from a portion of insertion portion 114 of second medical device 104.

Next, as shown in FIG. 4D, a portion of coupling device 106 may be manipulated to help secure second medical device 104 to first medical device 102. For example, with a portion of insertion portion 114 positioned within slot 152, gasket slot 160 (FIGS. 3A and 3B), and slot 176, as shown in FIG. 4C, the user may rotate second portion 134 of coupling device 106 in a first direction (e.g., clockwise or counterclockwise about a longitudinal axis of second portion 134) to rotate second portion 134 relative to first portion 132. For example, the user may rotate or otherwise manipulate grip portion 174 with hand 182, while maintaining the position of first medical device 102 with hand 180.

The rotation of second portion 134 may screw threaded portion 172 into threading 156, for example, helping to secure second portion 134 relative to first portion 132. Additionally, the rotation may help to reduce the size of opening 154 of device receiving portion 150 of first portion 132, which may, in turn, reduce the size and/or changing shape of gasket 158, for example, from the configuration shown in FIG. 3A to the configuration shown in FIG. 3B. For example, the rotation may engage threaded portion 172 with threading 156, and the engagement may cause device receiving portion 150 to reduce in size. Specifically, device receiving portion 150, in the unengaged configuration, may be larger than threaded portion 172, and thus device receiving portion 150 forms slot 152. As threaded portion 172 of second portion 134 engages with threading 156 of first portion 132, the size of device receiving portion 150 may reduce in size, which reduces the size of slot 152. Reducing the size of device receiving portion 150 also reduces the size and/or changes the shape of gasket 158, reducing the size of gasket slot 160 (e.g., transitioning from the configuration of FIG. 3A to the configuration of FIG. 3B). Furthermore, reducing the size and/or changing the shape of gasket 158 may help to secure insertion portion 114 to device receiving portion 150 of coupling device 106, thus coupling second medical device 104 to first medical device 102.

Additionally, although not shown, the user may rotate second portion 134 of coupling device 106 in a second direction (e.g., counterclockwise or clockwise). Rotating second portion 134 in the second direction may loosen or enlarge the size of opening 154 of device receiving portion 150 of first portion 132, and for example, enlarge the size and/or change the shape of gasket 158. In these aspects, the user may rotate second portion 134 in the second direction to adjust the position, arrangement, or configuration (e.g., longitudinally, rotatably, etc.) of second medical device 104 relative to first medical device 102, to replace second medical device 104 with a different medical device, or otherwise adjust aspects of medical system 100. In some aspects, the user may loosen the coupling of coupling device 106 around insertion portion 114 to distally extend and/or proximally retract insertion portion 114, for example, relative to port 116 and thus relative to a distal end (not shown) of insertion portion 110 of first medical device 102.

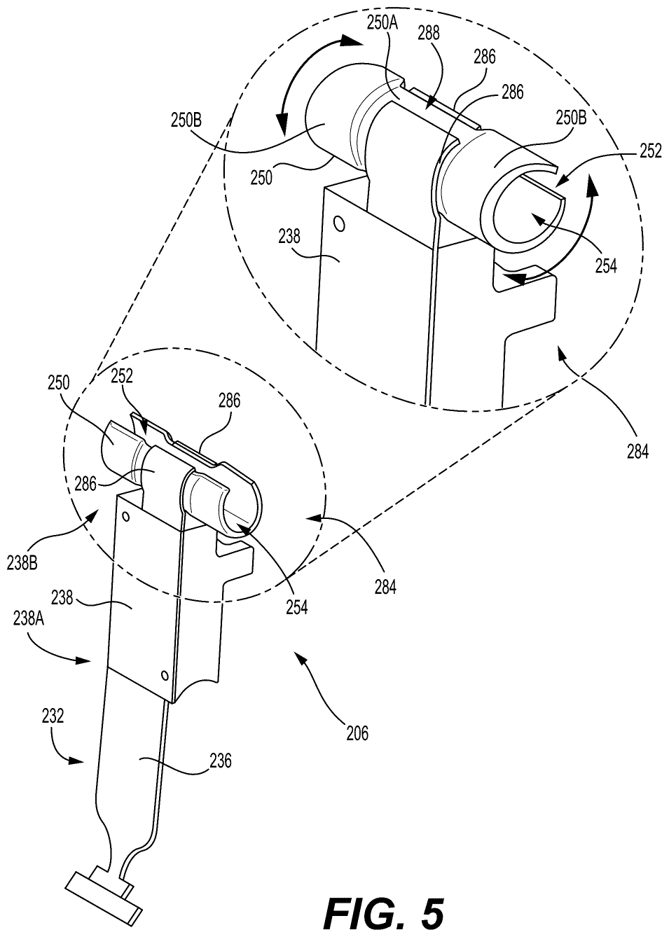

FIG. 5 illustrates a perspective and enlarged view of another coupling device 206, for example, to rotatably couple a first medical device and a second medical device, according to aspects of this disclosure. Coupling device 206 is similar to coupling device 106, except as noted below.

Coupling device 206 includes a first portion 232 and a second portion (not shown). First portion 232 includes a strap 236 and a main body 238, with strap 236 extending from a first end 238A of main body. Strap 236 may be wrapped around a portion of a first medical device and coupled to main body 238 to couple first portion 232 to the first medical device, as discussed above. First portion 232 also includes a receiving end 284, for example, on a second end 238B of main body 238 that is opposite to first end 238A. Receiving end 284 may include two arms 286, including an opening 288 (see enlarged inset) between ends of arms 286. First portion 232 may also include a device coupling portion 250, which may be, for example, separable and couplable from main body 238. Device coupling portion 250 may be generally cylindrical. Device coupling portion 250 includes a slot 252 and an opening 254. Although not shown in FIG. 5, slot 252 and opening 254 may receive and/or be otherwise couplable to a gasket, a second portion of coupling device 206, and a portion (e.g., an insertion portion) of a second medical device, having any of the properties discussed above for coupling device 106.

Device receiving portion 250 may include an indented portion 250A, for example, between two extended portions 250B. Indented portion 250A may be generally centrally positioned along a longitudinal length of device receiving portion 250. Additionally, indented portion 250A may be received within arms 286 to couple device receiving portion 250 within opening 288 of receiving end 284 of main body 238. In these aspects, device receiving portion 250 may be rotatable relative to main body 238, for example, clockwise and/or counterclockwise. For example, device receiving portion 250 may be rotatable within arms 286, as discussed below.

First portion 232 may be coupled to a second portion (not shown), as discussed above. For example, first portion 232 may include a gasket, for example, within opening 254, and first portion 232 may also include an internal threading or grooving (not shown). The second portion may include a threaded portion, such that coupling the second portion to first portion 232 includes screwing or otherwise coupling the threaded portion of the second portion to the internal threading of first portion 232. The coupling may also reduce the size of opening 254, and corresponding change the size and/or shape the gasket to help secure the portion of the second medical device within coupling device 206. Additionally, device receiving portion 250 may be rotatable relative to main body 238 without adjusting (e.g., loosening) the coupling between the second portion and first portion 232. For example, indented portion 250A of device receiving portion 250 may be rotatable relative to arms 286. In these aspects, the secondary medical device may be rotatable relative to the primary medical device without uncoupling the secondary medical device from coupling device 206.

Various aspects discussed herein may help reduce procedure time, increase tissue treatment effectiveness, reduce the risks to the subject, etc. Various aspects of this disclosure may allow for one or more users to use multiple endoscopic instruments for the resection of tissue, or otherwise provide treatment to the subject. In some instances, as discussed herein, a secondary medical device may be coupled to and/or inserted through a portion of a primary medical device in order to allow one user to hold and/or manipulate both the primary medical device and the secondary medical device, for example, to treat a treatment site. The user may couple different secondary medical devices to the primary medical device, as the coupling device may accommodate and/or be secured to different sizes and/or shapes of portions of secondary medical devices, for example, via the flexible gasket. Moreover, the coupling device may be coupled to different sizes and/or shapes of portions of primary medical devices, for example, via the connector or strap.

Additionally, as discussed herein, coupling the secondary medical device to the primary medical device and/or inserting the secondary medical device through the primary device may not limit the movement of the secondary medical device relative to primary medical device. For example, the user may loosen the coupling of the portions of the coupling device, and adjust the position of the secondary medical device relative to the primary medical device (e.g., to extend or retract the insertion portion of the secondary medical device relative to the insertion portion of the primary medical device). As a result, the user may experience less fatigue, may experience a larger range of motion, etc. Furthermore, the secondary medical device may be uncoupled from the primary medical device, for example, for operation by another user, and may later be re-coupled to the primary medical device. Alternatively or additionally, the secondary medical device may be uncoupled from the primary medical device, and another secondary medical device may be coupled to the primary medical device and operated by the user.

It also should be understood that one or more aspects of any of the medical devices, systems, and methods described herein may be used for manipulating, cutting, grabbing, ligating, biopsying, stenting, controlling bleeding, and/or otherwise treating tissue or a treatment site in any part of the human body. For example, any of the medical devices described herein may be used in medical procedures such as for EMR, ESD, a NOTES procedure, a stent procedure, an ERCP procedure, a POEM procedure, a hemostasis procedure, a pulmonary nodule biopsy procedure, cancer treatment, kidney or bladder biopsies or resections, and/or other procedures where removal, resection, dissection, fulguration, ablation, manipulation and/or any other treatment of tissue is needed.

Although the exemplary embodiments described above have been disclosed in connection with medical devices for manipulating and cutting human tissue through one or more working channels of a medical device, a natural orifice, or by incision, a person skilled in the art will understand that the principles set out above can be applied to any medical device or medical method and can be implemented in different ways without departing from the scope of the disclosure as defined by the claims. In particular, constructional details, including manufacturing techniques and materials, are well within the understanding of those of skill in the art and have not been set out in any detail here. These and other modifications and variations are well within the scope of this disclosure and can be envisioned and implemented by those of skill in the art.

Moreover, while specific exemplary embodiments may have been illustrated and described collectively herein, it should be appreciated that any subsequent arrangement designed to achieve the same or similar purpose may be substituted for the specific embodiments described and shown herein. This disclosure is intended to cover any and all subsequent adaptations or variations of various embodiments. Combinations of the above embodiments, and other embodiments not specifically described herein, will be apparent to those of skill in the art upon reviewing the description.

While principles of the disclosure are described herein with reference to illustrative aspects for particular applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

Other exemplary embodiments of this disclosure will be apparent to those skilled in the art from consideration of the specification and practice of the exemplary embodiments disclosed herein. It is intended that the specification and examples be considered as exemplary only, and departures in form and detail may be made without departing from the scope and spirit of this disclosure as defined by the following claims.

We claim:

1. A coupling device for a medical system, comprising:
a first portion, including:
a main body having a device receiving portion that includes a first slot and an opening, wherein a portion of the opening includes an internal threading; and
a connector, wherein the connector extends from the main body and is configured to couple a first medical device to the first portion; and
a second portion, wherein the second portion includes a second slot configured to receive a portion of a second medical device, and wherein the second portion includes a threaded portion,
wherein the first portion and the second portion are removably couplable by coupling the threaded portion of the second portion to the internal threading of the first portion to removably couple the second medical device to the first medical device.

2. The coupling device of claim 1, further comprising a gasket positioned within the opening of the first portion.

3. The coupling device of claim 2, wherein the gasket includes a gasket slot extending longitudinally through a portion of the gasket.

4. The coupling device of claim 3, wherein the gasket is configured to transition between at least a first configuration and a second configuration, wherein, in the first configuration, the gasket slot includes a first width, and wherein, in the second configuration, the gasket slot includes a second width that is less than the first width.

5. The coupling device of claim 4, wherein the gasket includes a gasket opening configured to receive a portion of the second medical device.

6. The coupling device of claim 1, wherein the second portion includes a grip portion adjacent to the threaded portion.

7. The coupling device of claim 6, wherein the second portion includes an end portion adjacent to the grip portion on an opposite side from the threaded portion, and wherein the end portion is wider than the grip portion.

8. The coupling device of claim 7, wherein the second slot extends longitudinally through the end portion, the grip portion, and the threaded portion of the second portion.

9. The coupling device of claim 1, wherein the connector extends from a first end of the main body of the first portion, and wherein the device receiving portion extends from a second end of the main body opposite to the first end.

10. The coupling device of claim 9, wherein the main body includes a connector receiving portion that includes an opening and two extensions, with the opening positioned between the two extensions.

11. The coupling device of claim 10, wherein the connector includes a narrowed portion and a widened portion, and wherein the narrowed portion is configured to be received within the opening of the connector receiving portion to couple the first portion of the coupling device to the first medical device.

12. The coupling device of claim 11, wherein the connector is formed of an at least partially flexible or elastic material.

13. The coupling device of claim 12, wherein the widened portion of the connector includes a grip surface.

14. The coupling device of claim 1, wherein the device receiving portion is rotatable relative to the main body of the first portion.

15. The coupling device of claim 1, wherein the first portion is configured to be coupled to an insertion portion of the first medical device, and wherein the second portion is configured to be coupled to an insertion portion of the second medical device.

16. A medical system, comprising:
a first medical device, including a first handle and a first insertion portion, wherein the first handle includes a port;
a second medical device, including a second handle and a second insertion portion, wherein at least a portion of the second insertion portion is configured to be inserted into the port; and
a coupling device, wherein the coupling device includes:
a first portion, including:
a main body including a device receiving portion, wherein the device receiving portion includes a first slot and an opening, and wherein a portion of the opening includes an internal threading; and
a connector, wherein the connector extends from the main body and is configured to couple a first medical device to the first portion;
a gasket positioned within the opening of the first portion, wherein the gasket includes a gasket slot aligned with the slot in the device receiving portion; and
a second portion, wherein the second portion includes a second slot configured to receive a portion of a second medical device, and wherein the second portion includes a threaded portion,
wherein the first portion and the second portion are removably couplable by coupling the threaded portion of the second portion to the internal threading of the first portion to (1) removably couple the second medical device to the first medical device, and (2) reduce a size of the gasket.

17. The medical system of claim 16, wherein the second portion includes (1) a grip portion adjacent to the threaded portion, and (2) an end portion adjacent to the grip portion that is on an opposite side from the threaded portion and is wider than the grip portion, wherein the second slot extends longitudinally through the end portion, the grip portion, and the threaded portion.

18. The medical system of claim 16, wherein the device receiving portion of the first portion is rotatable relative to the main body of the first portion.

19. A coupling device, comprising:
a first portion, including:
a main body, including a device receiving portion that includes a first slot and an opening; and
a connector, wherein the connector extends from the main body and is configured to couple a first medical device to the first portion;
a gasket, wherein the gasket is positioned within the opening of the first portion, wherein the gasket includes a gasket slot aligned with the slot in the device receiving portion; and
a second portion, wherein the second portion includes a second slot configured to receive a portion of a second medical device,
wherein the first portion and the second portion are removably couplable to removably couple the second medical device to the first medical device, wherein the first slot and the opening in the device receiving portion of main body of the first portion are both configured to reduce in size to secure the second portion to the first portion.

20. The coupling device of claim 19, wherein the device receiving portion is rotatable relative to the main body of the first portion.
